(19) United States
(12) Patent Application Publication
(10) Pub. No.: US 2022/0028487 A1
(43) Pub. Date: Jan. 27, 2022

(54) DEEP LEARNING-BASED METHOD FOR PREDICTING BINDING AFFINITY BETWEEN HUMAN LEUKOCYTE ANTIGENS AND PEPTIDES

(71) Applicant: Shenzhen NeoCura Biotechnology Corporation, Shenzhen (CN)

(72) Inventors: Yilin YE, Shenzhen (CN); Ji WAN, Shenzhen (CN); Jian WANG, Shenzhen (CN); Yunwan XU, Shenzhen (CN); Youdong PAN, Shenzhen (CN); Yi WANG, Shenzhen (CN); Qi SONG, Shenzhen (CN)

(73) Assignee: Shenzhen NeoCura Biotechnology Corporation, Shenzhen (CN)

(21) Appl. No.: 17/148,589

(22) Filed: Jan. 14, 2021

(30) Foreign Application Priority Data
Jul. 27, 2020 (CN) 202010732369.7

(51) Int. Cl. G16B 20/30; C12Q 1/6881; C07K 14/74; G06F 17/18

(52) U.S. Cl. CPC G16B 20/30; C12Q 1/6881; C07K 2317/92; G06F 17/18; C07K 14/70539

(57) ABSTRACT

A deep learning-based method for predicting a binding affinity between human leukocyte antigens (HLAs) and peptides includes: step S101: encoding HLA sequences; step S102: constructing a sequence of an HLA-peptide pair; step S103: constructing an encoding matrix of the HLA-peptide pair; step S104: constructing an affinity prediction model for HLA-peptide binding. The new method considers the effects of the protein sequences of HLAs and the sequences of the peptides on affinity strength and develops a deep learning-based method for predicting a binding affinity between HLAs and peptides.

### CROSS REFERENCE TO THE RELATED APPLICATIONS

This application is based upon and claims priority to Chinese Patent Application No. 202010732369.7, filed on Jul. 27, 2020, the entire contents of which are incorporated herein by reference.

### TECHNICAL FIELD

The present invention relates to the technical fields of immunotherapy and artificial intelligence, and in particular to a deep learning-based method for predicting a binding affinity between human leukocyte antigens and peptides.

### BACKGROUND

Currently, the binding of human leukocyte antigens (HLAs) to peptides plays a critical role in the presentation of epitope peptides to the cell surface and activation of the subsequent T-cell immune response. Predicting the binding affinity between HLAs and peptides by constructing a machine-learning model has been successfully applied to target selection for immunotherapy. Generally, methods for predicting HLA-peptide binding can be divided into antigen subtype-specific methods and pan-antigen subtype methods. Antigen subtype-specific methods require the construction of a prediction model for each HLA subtype, while pan-HLA subtype methods can perform affinity prediction between all HLA subtypes and peptides by integrating the core region of HLA for encoding. In the past few years, the experimental data and machine-learning algorithms of HLA-peptide binding have improved the prediction accuracy of binding affinity. The prediction accuracy for class I HLA-C requires to be further improved, however, due to the bias vectors of experimental data of existing methods (compared with class I HLA-A and HLA-B, the amount of experimental data for class I HLA-C is relatively small). Meanwhile, the length of peptides binding to class I HLAs is 8-15 amino acids, and the prediction accuracy of existing algorithms for relatively long peptides (12-15 amino acids) is much lower than that for short peptides, therefore, it is of great clinical significance to develop a more accurate prediction algorithm for the binding affinity between HLAs and peptides.

### SUMMARY

In view of the above-mentioned shortcomings, the present invention develops a deep learning- based method for predicting a binding affinity between human leukocyte antigens (HLAs) and peptides, taking into account the effects of the protein sequences of HLAs and the sequences of peptides on affinity strength.

The embodiment of the present invention provides a deep learning-based method for predicting a binding affinity between HLAs and peptides, including:

step S101: encoding HLA sequences;

step S102: constructing a sequence of an HLA-peptide pair;

step S103: constructing an encoding matrix of the HLA-peptide pair;

step S104: constructing an affinity prediction model for HLA-peptide binding.

Preferably, step S104: constructing an affinity prediction model for HLA-peptide binding, includes:

step S201: capturing information of the HLA-peptide sequence;

step S202: assigning weights to amino acids from a plurality of perspectives;

step S203: calculating an affinity between HLA and peptides.

Preferably, step S201: capturing information of the HLA-peptide sequence, includes:

treating each of the amino acids in the HLA-peptide sequence as a node in the HLA sequences;

sequentially sending encoding vectors of nodes into a bidirectional long short-term memory network; the bidirectional long short-term memory network can perform a feature learning on the HLA-peptide sequence according to a forward order and a reverse order of the HLA-peptide sequence, respectively.

Preferably, step S202: assigning weights to amino acids from a plurality of perspectives, includes:

mapping features of the HLA-peptide sequence to a plurality of feature spaces by a multi-head attention mechanism, and calculating attention weights of each of the amino acids in each of the plurality of feature spaces respectively to quantify an importance of each of the amino acids to an association of the HLA sequences with the peptides.

In a plurality of subspaces, the attention weights of each of the amino acids in each of the plurality of feature spaces can be obtained. In order to integrate the weights in the plurality of feature spaces, a convolution neural network with a filter size of head *1*1 is used to assign a weight to each of the feature spaces separately, and then, a weighted summation is performed on a plurality of attention weights of each of the amino acids, respectively, to obtain importance vectors of the sequences, the formula is as follows:

$$W = [ {w}_{1} , {w}_{2} , … , {w}_{head} ]$$

$$importance = {\sum }_{h}^{head} {w}_{h} \cdot {x}_{h}$$

where, W is a filter matrix of the convolution neural network, w^{h} is a weight corresponding to an h-th feature space, and xₕ is an attention weight vector of each of the amino acids in the h-th feature space.

Preferably, step S203: calculating an affinity between HLA sequences and peptides, includes:

integrating feature representations by two fully connected layers, and using a Sigmoid function to obtain a value between 0-1 as an affinity score of HLA sequence-peptide pairs, the formula is as follows:

temp1=Tanh(out·W₁+b₁)

x=Sigmoid(temp1·W₂+b₂)

where, W₁ and W₂ are weight matrices of the two fully connected layers respectively, b₁ and b₂ are bias vectors of the two fully connected layers respectively, and Tanh represents a hyperbolic tangent function.

Preferably, step S101: encoding HLA sequences, includes:

using pseudo sequences of an HLA core region to represent HLA subtypes.

Preferably, step S102: constructing a sequence of an HLA-peptide pair, includes:

splicing the pseudo sequences and the corresponding peptide sequences into a whole to form an amino acid sequence with a length of 42-49.

Preferably, step S103: constructing an encoding matrix of the HLA-peptide pair, includes:

encoding each of the amino acids in the HLA-peptide sequence using a BLOSUM62 matrix to form the encoding matrix with a dimension of lseq*20, where the lseq represents the length of the sequence;

or,

encoding each of the amino acids in the HLA-peptide sequence using One-Hot vectors to form the encoding matrix.

Compared with the prior art, the solution of the present invention has the following advantages.

1. In principle, the deep learning algorithm used in the present invention can facilitate the learning of the deeper and more original sequence representation of the HLA-peptide pair, thus laying a solid foundation for providing an accurate and reliable affinity prediction.

2. The present invention adopts a deep neural network-based bidirectional long short-term memory network, and achieves the affinity prediction between most HLA-A, HLA-B and peptides with a plurality of lengths through a single model. Moreover, the affinity prediction between HLA-C and peptides achieves the same stability as that between HLA-A, HLA-B and peptides even if there is less research data on HLA-C. Experiments prove that the prediction performance of the present algorithm on class I HLA-A, HLA-B and HLA-C and peptide sequences with a length of 8-15 amino acids is better and more stable compared with other prediction algorithms.

3. Through the multi-head attention mechanism in the present algorithm, the importance of each of the amino acids in the sequence is evaluated from a plurality of perspectives. Finally, when predicting the affinity strength, the network can have a comprehensive understanding of the whole sequence, and selectively enhance or weaken the information of each site, so as to obtain more accurate and stable affinity prediction results. Meanwhile, the contribution of different amino acid positions in the sequence to the affinity strength can also be displayed in this process, so as to more accurately understand and analyze the interaction mechanism between them.

Other features and advantages of the present invention will be illustrated in combination with the specification and, in part, will be apparent from the description or understood by the implementation of the present invention. The objective and other advantages of the present invention can be achieved and obtained by the description, claims and the structure specially pointed out in the drawings.

The technical solution of the present invention is further described in detail with the drawings and embodiments.

### BRIEF DESCRIPTION OF THE DRAWINGS

The drawings are used to provide a further understanding of the present invention and form a part of the specification. They are used to explain the present invention together with the embodiments of the present invention and do not constitute a limitation of the present invention. In the drawings:

FIG. 1 is a schematic diagram showing a deep learning-based method for predicting a binding affinity between HLAs and peptides in the embodiment of the present invention;

FIG. 2 is a schematic diagram showing an algorithm implementation of a deep learning-based method for predicting a binding affinity between HLAs and peptides in the embodiment of the present invention.

### DETAILED DESCRIPTION OF THE EMBODIMENTS

Preferred embodiments of the present invention will now be described with reference to the drawings. It should be understood that the preferred embodiments described herein are only used to illustrate and explain the present invention, and are not intended to limit the present invention.

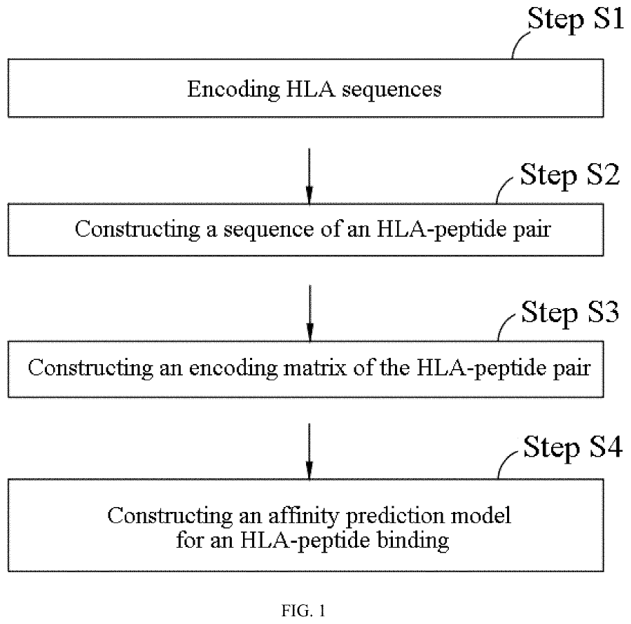

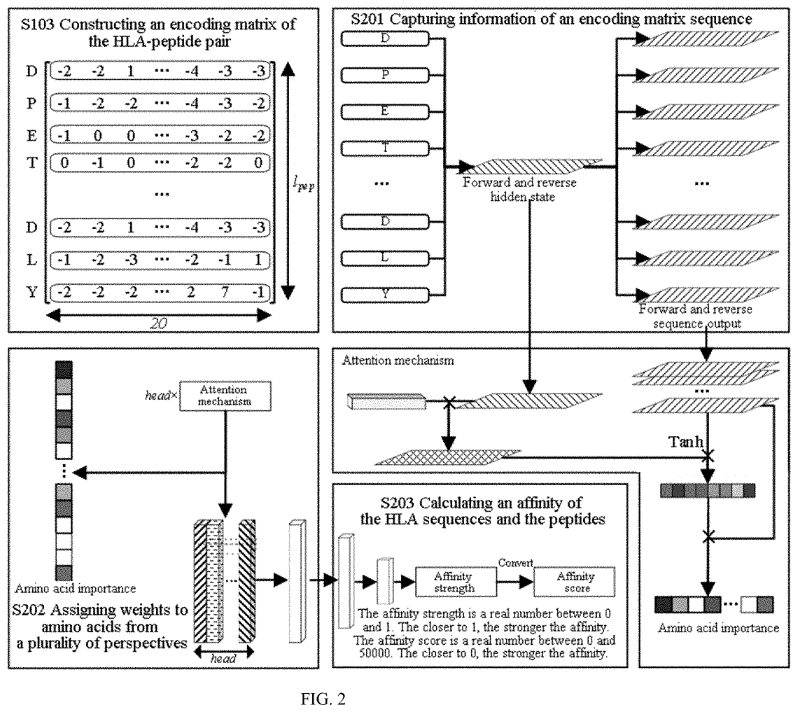

FIG. 1 and FIG. 2 show an embodiment of the present invention. A deep learning-based method for predicting a binding affinity between HLAs and peptides includes the following steps.

Step S101, HLA sequences are encoded.

In order to facilitate computer calculation, pseudo sequences of an HLA core region are used to represent HLA subtypes (http://www.cbs.dtu.dk/services/NetMHCpan/). Each of the pseudo sequences of HLAs is a character string sequence with a length of 34, in which each character represents an amino acid.

For example, a pseudo sequence of HLA-A*0101 is “YFAMYQENMAHTDANTLYIIYRDYTWVARVYRGY” (as shown in SEQ ID NO.1).

In this step, the element of the used pseudo sequences of the HLA core region is consistent with the peptide sequences, which provides convenience for subsequent splicing and encoding of HLAs and peptide sequences.

Step S102, a sequence of an HLA-peptide pair is constructed.

Peptides of 8-15 amino acids in length are used for subsequent analysis. The pseudo sequences obtained in the previous step and the corresponding peptide sequences are spliced into a whole to form an HLA-peptide sequence with a length of 42-49, which is used for the construction of a pan-antigen subtype model.

Unlike most algorithms in the prior art that are required to construct multiple models for different HLAs, our algorithm splices the HLA sequences and peptide sequences through a unified model for analysis, which can more comprehensively consider the relationship between the HLA sequences and peptide sequences. Therefore, the HLAs supported by the present model is more extensive, and HLAs newly discovered in the future is also supported without retraining the corresponding model.

Step S103, an encoding matrix of the HLA-peptide pair is constructed.

Then, in order to calculate the spliced sequence though deep learning network, it is needed to encode the spliced sequence digitally. BLOSUM62 matrix is an amino acid substitution scoring matrix used for sequence alignment in bioinformatics, which represents the substitution scores of 20 amino acids. Therefore, the BLOSUM62 matrix is extracted by row as feature vectors of corresponding amino acids. For example, the BLOSUM62 encoding of amino acid “Y” is “−2, −2, −2, −3, −2, −1, −2, −3, 2, −1, −1, −2, −1, 3, −3, −2, −2, 2, 7, −1”. Then, each of the amino acids in the HLA-peptide sequence obtained above is encoded to form a feature encoding matrix with a dimension of lseq*20, where the lseq represents the length of the sequence.

Alternatively, the amino acids can be encoded through One-Hot vectors. Since a total of 20 amino acids are involved, One-Hot is encoded as a vector with a length of 20. Each amino acid is corresponded to each position in the vector. The present amino acid is located at position 1 and the rest is 0. If amino acid “Y” is located at the 19th position, then its One-Hot vector is: “0, 0, 0, 0, 0, 0, 0, 0, 0, 0, 0, 0, 0, 0, 0, 0, 0, 0, 1, 0”.

Compared with other encoding methods (such as One-Hot encoding), the BLOSUM62 encoding carries more knowledge from a biological background, and can better express the potential relationship between amino acids in limited coding bits.

Step S104: an affinity prediction model for HLA-peptide binding is constructed. Based on the established prediction model, the binding affinity between HLAs and peptides is predicted. This step includes step S201: capturing information of the HLA-peptide sequence.

The HLA sequence-peptide encoding is analyzed by a bidirectional long short-term memory network from a sequence perspective. Each of the amino acids in the sequence is regarded as a node in the sequence, then encoding vectors of nodes are successively sent into the bidirectional long short-term memory network. The bidirectional long short-term memory network can perform feature learning on the sequence according to a forward order and a reverse order of the sequence, respectively. The purpose of doing this is to capture the context feature information of the sequence at the same time, so that the network can better learn the encoding representation of the HLA-peptide sequence.

A PyTorch framework is taken as an example to illustrate the learning process of the network.

First, a definition of the bidirectional long short-term memory network is given:

self.LSTM=nn.LSTM(input_size=parms_Net[‘len_acid’],
hidden_size=self.HIDDEN_SIZE,
num_layers=self.LAYER_NUM,
bidirectional=True)

where, input_size specifies a number of amino acids in the HLA-peptide sequence. Hidden_size specifies how large a parameter analysis data should be used in the bidirectional long short-term memory network, num_layers specifies a number of network layers to be used, and bidirectional specifies to use the bidirectional long short-term memory network to analyze the data.

Subsequently, sequence features learned by the bidirectional long short-term memory network are obtained by out^{lstm}, hidden^{lstm}=self.LSTM(x), where x is an encoded feature matrix.

Previous algorithms for predicting affinity between HLAs and peptides require peptides with different lengths to be filled to a unified length for prediction, which causes computational resources to be wasted on a large number of meaningless filling characters. Our algorithm can directly support sequence analysis of different lengths due to the flexible sequence analysis characteristic of the bidirectional long short-term memory network, while saving computing resources, the network can focus more accurately on the effective information of the sequence itself.

Step S202: weights are assigned to amino acids from a plurality of perspectives.

Sequence features are mapped to a plurality of feature subspaces by a multi-head attention mechanism, and attention weights of each of the amino acids in each of the plurality of feature subspaces are calculated respectively to quantify an importance of each of the amino acids to an association of the HLA sequences with the peptides. Specifically, this process is realized by the following formula:

$${W}_{i}^{atten} - {hidden}^{lstm} \cdot {W}_{i}^{project}$$

$${Context}_{i} = {W}_{i}^{atten} \cdot {( Tanh ( {out}^{lstm} ) )}^{T}$$

$$total = {\sum }_{k = 0}^{h} {Context}_{k}$$

$${importance}_{i} = \frac{{Context}_{i}}{total}$$

$${Head}_{i} = {importance}_{i} \cdot {out}^{lstm}$$

Firstly, weights hidden^{lstm} in the bidirectional long short-term memory network are projected into several different subspaces by the network through several projection matrices Wᵢ^{project} to obtain new weights Wᵢ^{atten}; out^{lstm} is an output of the bidirectional long short-term memory network, which is transformed by the hyperbolic tangent (Tanh) function and multiplied by Wᵢ^{atten} to obtain context vectors Contextᵢ, which represents a context representation of a bidirectional sequence representation in different spaces.

In order to calculate the importance of each of the amino acids in the original sequence at a certain perspective, the context vectors in all spaces are required to be calculated for summation, which is recorded as total. Then, a ratio of a context vector Contextᵢ and total in any space is an importance of an amino acid in this space, which is recorded as importanceᵢ. importanceᵢ is a vector with the same length as the sequence, where each bit represents the importance of the corresponding amino acid in the i-th space, the closer to 1 indicates the more important the amino acid, and the closer to 0 indicates the multi-head attention mechanism tries to shield the information from the amino acid in the i-th space.

Finally, the weighted representation Headᵢ of the original sequence in the i-th space is the product of the output out^{lstm} of the bidirectional long short-term memory network and importanceᵢ. According to the previous definition, the information from the important position of the sequence will be weighted by a weight close to 1, while the unimportant position will be shielded by being assigned with a weight close to 0.

In a plurality of subspaces, several different weighted sequence feature representations can be obtained. In order to integrate the weights of each of the feature spaces, a convolution neural network with a filter size of head *1*1 is used to assign a weight to each of the feature spaces separately, and then, a weighted summation is performed on a plurality of weights of each of the amino acids, respectively, to obtain the importance of the amino acid, the formula is as follows:

$$W = [ {w}_{1} , {w}_{2} , … , {w}_{head} ]$$

$$importance = {\sum }_{h}^{head} {w}_{h} \cdot {x}_{h}$$

where, W is a filter matrix of the convolution neural network, wₕ is a weight corresponding to an h-th feature space, and xₕ is an attention weight vector of each of the amino acids in the h-th feature space.

The code is as follows:

self.MixHead=nn.Conv2d(in_channels=self.head,out_channels=1,kernel_size=1)

importance=self.MixHead(x)

where, in_channels specifies that a depth of convolution is consistent with a number of subspaces mentioned above, out_channels specifies that an output depth of convolution is 1, kernel_size specifies that a size of the filter is 1*1, and x is an output of the multi-head attention mechanism.

This step focuses not only on the sequence itself, but also on the amino acids that play an important role in the sequence. Therefore, the importance of each position in the sequence is evaluated from a plurality of feature spaces via the multi-head attention mechanism, and the information of amino acids located on those important positions is concentrated. Therefore, consistent and stable prediction performance can be achieved on different lengths and different types of sequences.

Step S203: an affinity between HLA sequences and peptides is calculated.

The above-mentioned feature representations are integrated by two fully connected layers, and a Sigmoid function is used to obtain a value between 0-1 as an affinity score of an HLA sequence-peptide pair, the formula is as follows:

temp1=Tanh(out·W₁+b₁)

x=Sigmoid(temp1·W₂+b₂)

where, W₁ and W₂ are weight matrices of the two fully connected layers respectively, and b₁ and b₂ are bias vectors of the two fully connected layers respectively. In order to increase a nonlinear expression ability of the model, a hyperbolic tangent (Tanh) transformation is further added between the two fully connected layers. The Sigmoid function is responsible for converting predicted values into decimals between 0-1, indicating the affinity score of the HLA sequence-peptide pair. The closer to 1, the stronger the affinity.

The code is as follows:

out_fc1=nn.Linear(in_features=2*self HIDDEN_SIZE,out_features=self.HIDDEN_SIZE)

out_fc2=nn.Linear(in_features=self.HIDDEN_SIZE,out_features=1)

temp1=out_fc 1(out)

temp1=torch. Tanh(temp1)

temp2=out_fc2(temp1)

x=torch.sigmoid (temp)

If a specific affinity value is needed, the affinity score only needs to be converted:

Affnity=50000^{1-x}

where, x is an affinity score, and Affnity is an affinity strength. The closer to 0, the stronger the affinity. Generally, the affinity strength within 500 indicates that there is a relatively strong affinity between the HLA sequences and peptides.

Obviously, those skilled in the art can make various modifications and variations to the present invention without departing from the spirit and scope of the present invention. In this regard, if these modifications and variations of the present invention fall within the scope of claims of the present invention and the equivalent technologies, the present invention also intends to include these modifications and variations.

What is claimed is:

1. A deep learning-based method for predicting a binding affinity between human leukocyte antigens (HLAs) and peptides, comprising:
step S101: encoding HLA sequences;
step S102: constructing a sequence of an HLA-peptide pair;
step S103: constructing an encoding matrix of the HLA-peptide pair;
step S104: constructing an affinity prediction model for an HLA-peptide binding.

2. The deep learning-based method according to claim 1, wherein step S104: constructing the affinity prediction model for the HLA-peptide binding comprises:
step S201: capturing information of an HLA-peptide sequence;
step S202: assigning weights to amino acids in the HLA-peptide sequence from a plurality of perspectives;
step S203: calculating an affinity between the HLA sequences and the peptides.

3. The deep learning-based method according to claim 2, wherein step S201: capturing the information of the HLA-peptide sequence comprises:
treating the amino acids in the HLA-peptide sequence as nodes in the HLA sequences;
sequentially sending encoding vectors of the nodes into a bidirectional long short-term memory network; wherein the bidirectional long short-term memory network performs a feature learning on the HLA-peptide sequence according to a forward order of the HLA-peptide sequence and a reverse order of the HLA-peptide sequence, respectively.

4. The deep learning-based method according to claim 2, wherein step S202: assigning the weights to the amino acids in the HLA-peptide sequence from the plurality of perspectives comprises:
mapping features of the HLA-peptide sequence to a plurality of feature spaces by a multi-head attention mechanism;
in a plurality of subspaces, obtaining a plurality of attention weights of each of the amino acids in each of the plurality of feature spaces;
assigning a weight to each of the feature spaces separately by a convolution neural network with a filter size of head *1*1, and then, performing a weighted summation on the plurality of attention weights of each of the amino acids, respectively, to obtain importance vectors of the HLA-peptide sequence, wherein a formula is as follows:
$$W = [ {w}_{1} , {w}_{2} , … , {w}_{head} ]$$
$$importance = {\sum }_{h}^{head} {w}_{h} \cdot {x}_{h}$$
wherein, W is a filter matrix of the convolution neural network, wₕ is the weight corresponding to an h-th feature space, and Xₕ is an attention weight vector of each of the amino acids in the h-th feature space.

5. The deep learning-based method according to claim 2, wherein step S203: calculating the affinity between the HLA sequences and the peptides comprises:
integrating feature representations by two fully connected layers, and using a Sigmoid function to obtain a value between 0-1 as an affinity score of the HLA-peptide pair, wherein a formula is as follows:
temp1=Tanh(out·W₁+b₁)
x=Sigmoid(temp1·W₂+b₂)
wherein, W₁ and W₂ are weight matrices of the two fully connected layers respectively, b₁ and b₂ are bias vectors of the two fully connected layers respectively, and Tanh represents a hyperbolic tangent transformation.

6. The deep learning-based method according to claim 1, wherein step S101: encoding the HLA sequences comprises:
using pseudo sequences of an HLA core region to represent HLA subtypes.

7. The deep learning-based method according to claim 6, wherein step S102: constructing the sequence of the HLA-peptide pair comprises:
splicing the pseudo sequences and peptide sequences corresponding to the pseudo sequences into a whole to form the HLA-peptide sequence with a length of 42-49.

8. The deep learning-based method according to claim 7, wherein step S103: constructing the encoding matrix of the HLA -peptide pair comprises:
encoding each of amino acids in the HLA-peptide sequence using a BLOSUM62 matrix to form the encoding matrix with a dimension of lseq*20, wherein the lseq represents the length of the HLA-peptide sequence;
or,
encoding each of the amino acids in the HLA-peptide sequence using One-Hot vectors to form the encoding matrix.


SEQUENCE LISTING (1 sequence)

SEQ ID NO: 1 - protein, 34 aa, Homo sapiens
YFAMYQENMAHTDANTLYIIYRDYTWVARVYRGY